(19) United States
(12) Patent Application Publication
(10) Pub. No.: US 2008/0138432 A1
(43) Pub. Date: Jun. 12, 2008

(54) Acute Inflammatory Condition Treatment

(75) Inventors: Arkady Mandel, Mississauga (CA); Anthony E. Bolton, Dublin (IE)

Correspondence Address: FOLEY & LARDNER LLP, 975 PAGE MILL ROAD, PALO ALTO, CA, 94304, US

(73) Assignee: Vasogen Ireland Limited

(21) Appl. No.: 11/632,865

(22) PCT Filed: Jul. 18, 2005
(86) PCT No.: PCT/CA05/01128

§ 371 Date: Aug. 16, 2007

Related U.S. Application Data
(60) Provisional application No. 60/589,947, filed on Jul. 20, 2004.

(51) Int. Cl. A61K 35/14; A61P 29/00

(52) U.S. Cl. 424/529

(57) ABSTRACT

This invention provides a method for prophylaxis or treatment of an acute inflammatory disorder, comprising administering to a patient an aliquot of the patient's blood extracted from the patient and treated ex vivo with at least two stressors selected from the group consisting of an oxidizing agent, an electromagnetic emission and elevated temperature.

### FIELD OF THE INVENTION

This invention relates to processes, medical treatments, and compositions for alleviating acute inflammatory conditions in mammalian patients.

### BACKGROUND OF THE INVENTION

“Acute inflammatory conditions” as the term is used herein, and in accordance with normal medical parlance, refers to inflammatory conditions having a rapid onset and severe symptoms. The duration of the onset, from a normal condition of the patient to one in which symptoms of inflammation are seriously manifested, is anything up to about 72 hours. Acute inflammatory conditions are to be contrasted with chronic inflammatory conditions, which are inflammatory conditions of long duration, denoting a disease showing little change or of slow progression. The distinction between acute and chronic conditions is well known to those in the medical professions, even if they are not distinguishable by rigid, numbers-based definitions.

It is known that many inflammatory conditions are associated with an abnormal secretion level of various cytokines in the mammalian body. Professional antigen-presenting cells (APCs), including dendritic cells and macrophages, actively capture and process antigens, clear cell debris, and remove infectious organisms and dying cells, including the residues from dying cells. During this process, APCs can stimulate the production of either inflammatory Th1 pro-inflammatory cytokines (IL-12, IL-1, TNF-α, IFN-γ, etc); or regulatory, Th2/Th3 anti-inflammatory cytokines (IL-10, IL-4, TGF-β etc) dominated responses, depending on the nature of the antigen or phagocytosed material and the level of APC maturation/activation.

The present invention addresses acute inflammatory disorder by a process involving subjection of blood to oxidizing environments such as ozone.

### BRIEF REFERENCE TO THE PRIOR ART

U.S. Pat. No. 3,715,430 Ryan relates to a method and apparatus for producing substantially pure oxygen having a controlled content of ozone and higher oxygen polymers. The purified oxygen gas is exposed to ultraviolet light in a wavelength of 2485 to 2537 angstrom units in order to produce 5 to 500 parts per million of ozone and higher oxygen polymers in the gas mixture. Ryan indicates that the gas produced in this manner is non-irritating to the human body and may be intravenously injected into the blood stream for therapeutic use.

U.S. Pat. No. 4,632,980 Zee et al. discloses a method of freeing blood and blood components of enveloped viruses by contacting the blood or blood product in an aqueous medium with an enveloped virus inactivating amount of ozone. The treatment is carried out at a temperature of 4° to 37° Celsius, and an ozone concentration of 1-100 ppm.

U.S. Pat. No. 4,831,268 Fisch et al. provides a method for the radiation of blood to prevent arteriosclerosis related heart and vascular diseases caused by disturbances in the fat exchange. The disclosed process involves irradiating the blood in a blood conducting tube with radiation having an intensity of from about 1 mW/cm² to 10 mW/cm² in a wavelength of from about 300 to 600 nm.

U.S. Pat. No. 4,968,483 Mueller et al. describes an apparatus for oxygenating blood, by treating an aliquot of a patient's blood extracorporeally, with an oxygen/ozone mixture and ultraviolet light, at a controlled temperature. The apparatus is proposed for use in haematological oxidation therapy.

U.S. Pat. No. 6,204,058 Bolton discloses a process for preparing an autovaccine for administration to an autoimmune disease-suffering mammalian patient to alleviate the patient's autoimmune disease symptoms, by treating an extracted blood aliquot extracorporeally by subjecting it to an immune system-modifying amount of ozone gas and ultraviolet radiation, where the treated blood aliquot has at least one specified feature such as increased numbers of leucocytes exhibiting a condensed apoptotic-like morphology.

### SUMMARY OF THE INVENTION

The present invention is based upon the discovery that blood treated with various stressors such as ozone, will, upon administration to a mammalian patient, cause a rapid decrease in the level of inflammatory cytokines such as TNF-α, IFN-γ and IL-12, the effects being significant within the first twelve hours after the administration of the treated blood. Accordingly, the treated blood may be used to treat acute inflammatory diseases and/or to delay and/or to ameliorate symptoms associated with such diseases.

In one aspect, the present invention provides a method of alleviating acute inflammatory conditions in a mammalian subject, comprising: (a) extracting an aliquot of blood from the subject; (b) treating the aliquot of blood ex vivo with at least two stressors selected from the group consisting of an oxidizing agent, an electromagnetic emission and elevated temperature; and (c) administering the aliquot of blood treated in step (b) to the subject.

A further aspect of the invention is the use in the preparation of a medicament for treating acute inflammatory conditions in a mammalian patient, of autologous blood which has been stressed ex vivo by subjection to at least two stressors selected from an oxidizing agent, electromagnetic emission, and elevated temperature.

The preparation of the treated blood for use in the present invention preferably comprises extracting from the subject an aliquot of blood of volume about 0.01 ml to about 400 ml, and contacting the aliquot of blood, extracorporeally, with an immune system-stimulating effective amount of ozone gas and an electromagnetic transmission.

The method for alleviating acute inflammatory conditions in a human subject, in accordance with a preferred embodiment of the present invention, comprises extracting from the patient an aliquot of blood of volume about 0.01 ml to about 400 ml, contacting the aliquot of blood, extracorporeally, with an immune system-stimulating amount of ozone gas and an electromagnetic emission, followed by administering the treated blood aliquot to the subject.

Following administration to the mammalian patient, the treated blood is believed to interact rapidly with the immune system resulting in the rapid development of an anti-inflammatory response, as evidenced by changes in cytokine profile.

### BRIEF DESCRIPTION OF THE DRAWINGS

FIG. 1 is a set bar graphs comparing the cytokine IL-1β mRNA expression (12 hour vs. 24 hour) from the draining lymph node of mice treated in the ICD model (as later described), Example 1 herein;

FIG. 2 is a similar graph for the cytokine IFN-γ, Example 1 herein;

FIG. 3 is a similar graph for the cytokine IL-12, Example 1 herein

### DESCRIPTION OF PREFERRED EMBODIMENTS

According to the present invention, blood from patients suffering from acute inflammatory conditions is treated, extracorporeally, with various stressors such as an electromagnetic emission or ozone (including combinations of stressors).

Exactly how the treatment operates following this re-injection is not currently fully understood. The following tentative explanation is offered for a better and more complete description of the invention, but is not to be considered as binding or limiting.

T-cells, which are one kind of lymphocyte and which play a significant role in the control of the immune system, include CD-8 cells, and CD-4 cells otherwise known as T-helper cells, further subdividable into Th1 and Th2 cells. The Th1 cells secrete pro-inflammatory cytokines such as interferon gamma (IFN-γ). The Th2 cells are considered to be regulatory cells and secrete regulatory cytokines, such as interleukin-4 (IL-4). A number of components of the treated blood of the present invention, possibly including HLA-DR and/or other MHC antigens released from the leucocyte cell surfaces, upregulate the Th2 cells in the patient's blood and/or locally at the site of the inflammation, thereby increasing the secretion of regulatory cytokines or having other effects.

A patient or a subject is identified as having a need for prophylaxis or treatment of an acute inflammatory disorder. A blood aliquot from the patient is prepared by exposing the aliquot to at least two stressors, in controlled amounts, the stressor being selected from among oxidizing agents such as ozone, ultraviolet radiation and elevated temperature. Combinations of all three of such stressors are preferred. The resulting blood aliquot, after such treatment, can be reinjected into the patient.

Preferably, the stressors to which the cells in the extracted blood aliquot are subjected are a temperature stress (blood temperature above body temperature), an oxidative environment such as a mixture of ozone and oxygen bubbled through the blood aliquot, and an electromagnetic emission, individually, in any combination, simultaneously or successively, but preferably simultaneously.

In general, from about 0.01 ml to about 400 ml of blood may be treated according to the invention. Preferred amounts are in the range of about 0.1 to about 200 ml. More suitably, the aliquot for treatment has a volume of from about 0.1 to about 100 mls, preferably 1 to about 50 ml and most preferably 5 to 15 ml. The method most preferably involves treating an aliquot of about 10 mls blood with ozone gas and an electromagnetic emission, then re-administering the treated blood to the patient by intramuscular injection.

It is preferred, according to the invention, to apply a temperature stress (blood temperature above or below body temperature), in addition to the electromagnetic emission stress and the oxidative stress. Preferably, all three of the aforementioned stressors are applied simultaneously to the aliquot under treatment, in order to ensure the appropriate modification to the blood. Care must be taken to utilize an appropriate level of the stressors to thereby effectively modify the blood to alleviate the acute inflammatory condition in the subject.

The temperature stressor warms the aliquot being treated to a temperature above normal body temperature or cools the aliquot below normal body temperature. The temperature is selected so that the temperature stressor does not cause excessive hemolysis in the blood contained in the aliquot and so that, when the treated aliquot is injected into a subject, alleviation of the acute inflammatory condition will be achieved. Preferably, the temperature stressor is applied so that the temperature of all or a part of the aliquot is up to about 55° C., and more preferably in the range of from about −5° C. to about 55° C.

In some preferred embodiments of the invention, the temperature of the aliquot is raised above normal body temperature, such that the mean temperature of the aliquot does not exceed a temperature of about 55° C., more preferably from about 40° C. to about 50° C., even more preferably from about 40° C. to about 44° C., and most preferably about 42.5±1° C.

In other preferred embodiments, the aliquot is cooled below normal body temperature such that the mean temperature of the aliquot is within the range of from about −5° C. to about 36.5° C., even more preferably from about 10° C. to about 30° C., and even more preferably from about 15° C. to about 25° C.

Alternatively, the blood sample is heated while being subjected to an electromagnetic emission until the blood reaches a predetermined temperature (preferably about 42.5±1° Celsius) at which point bubbling of ozone gas through the blood is commenced. The concurrent electromagnetic emission/ozone treatment is then maintained for a predetermined period of time, preferably about 3 minutes.

Another alternative method involves subjecting the blood to electromagnetic emission/ozone while heating to a predetermined temperature (preferably about 42.5±1° Celsius), then either ending the treatment once the predetermined temperature is reached, or continuing electromagnetic emission/ozone treatment for a further period of time, most preferably about 3 minutes.

The oxidative environment stressor can be the application to the aliquot of solid, liquid or gaseous oxidizing agents. Chemical oxidants such as hydrogen peroxide can be used. Preferably, it involves exposing the aliquot to ozone gas. More preferably, it involves exposing the aliquot to a mixture of medical grade oxygen and ozone gas, most preferably by bubbling through the aliquot, at the aforementioned temperature range, a stream of medical grade oxygen gas having ozone as a minor component therein. The ozone content of the gas stream and the flow rate of the gas stream should be selected such that the amount of ozone introduced to the blood aliquot, either on its own or in combination with other stressors, does not give rise to excessive levels of cell damage such that the therapy is rendered ineffective. Suitably, the gas stream has an ozone content of up to about 300 μg/ml, preferably up to about 100 μg/ml, more preferably about 30 μg/ml, even more preferably up to about 20 μg/ml, particularly preferably from about 10 μg/ml to about 20 μg/ml, and most preferably about 14.5±1.0 μg/ml. The gas stream is suitably supplied to the aliquot at a rate of up to about 2.0 litres/min, preferably up to about 0.5 litres/min, more preferably up to about 0.4 litres/min, even more preferably up to about 0.33 litres/min, and most preferably about 0.24±0.024 litres/min. The lower limit of the flow rate of the gas stream is preferably not lower than 0.01 litres/min, more preferably not lower than 0.1 litres/min, and even more preferably not lower than 0.2 litres/min.

The electromagnetic emission stressor is suitably applied by irradiating the aliquot under treatment from a source of an electromagnetic emission while the aliquot is maintained at the aforementioned temperature and while the oxygen/ozone gaseous mixture is being bubbled through the aliquot. Preferred electromagnetic emissions are selected from photonic radiation, more preferably UV, visible and infrared light, and even more preferably UV light. The most preferred UV sources are UV lamps emitting primarily UV-C band wavelengths, i.e. at wavelengths shorter than about 280 nm. Such lamps may also emit amounts of visible and infrared light. Ultraviolet light corresponding to standard UV-A (wavelengths from about 315 to about 400 nm) and UV-B (wavelengths from about 280 to about 315) sources can also be used. For example, an appropriate dosage of such UV light, applied simultaneously with the aforementioned temperature and oxidative environment stressors, can be obtained from lamps with a combined power output of from about 45-65 mW/cm². Up to eight such lamps surrounding the sample container holding the aliquot, with a combined output at 253.7 nm of 15-25 watts, operated at an intensity to deliver a total UV light energy at the surface of the blood of from about 0.025 to about 10 joules/cm², preferably from about 0.1 to about 3.0 joules/cm². Preferably, four such lamps are used.

The time for which the aliquot is subjected to the stressors can be from a few seconds to about 60 minutes. The time depends to some extent upon the chosen intensity of the electromagnetic emission, the temperature and the concentration of the rate at which the oxidizing agent is supplied to the aliquot. Some experimentation to establish optimum times may be necessary on the part of the operator, once the other stressor levels have been set. Under most stressor conditions, preferred times will be in the approximate range of from about 2 to about 5 minutes, more preferably around 3 minutes. The starting blood temperature and the rate at which it can be warmed or cooled to a predetermined temperature, tends to vary from subject to subject.

Warming is suitably by use of one or more infrared lamps placed adjacent to the aliquot container. Other methods of warming can also be adopted.

In the practice of the preferred process of the present invention, the blood aliquot (or the separated cellular fractions of the blood, or mixtures of the separated cells, including platelets, these various leucocyte-combinations, along with whole blood, being referred to collectively throughout as the “aliquot”) may be treated with the stressors using an apparatus of the type described in U.S. Pat. No. 4,968,483 Mueller. The aliquot is placed in a suitable, sterile, UV light-transmissive container, which is fitted into the machine. The UV lamps are switched on for a fixed period before the gas flow is applied to the aliquot providing the oxidative stress, to allow the output of the UV lamps to stabilize. The UV lamps are typically on while the temperature of the aliquot is adjusted to the predetermined value, e.g. 42.5±1° C. Then the oxygen/ozone gas mixture, of known composition and controlled flow rate, is applied to the aliquot, for the predetermined duration of up to about 60 minutes, preferably 2 to 5 minutes and most preferably about 3 minutes as discussed above, so that the aliquot experiences all three stressors simultaneously. In this way, blood is appropriately modified according to the present invention to achieve the desired effects.

Thus, the invention also provides a method of stimulating or activating the immune system in the a human by contacting about 0.01 ml to about 400 ml of blood from a human with an immune system-stimulating amount of ozone gas and ultraviolet radiation, followed by administering the treated blood to a human. It is believed that this stimulation or activation of the immune system may have the effect of treating acute inflammatory conditions or disorders. Similarly, the invention contemplates a method of treating an existing acute inflammatory condition or disorder in a human by contacting about 0.01 ml to about 400 ml of blood from a human with an immune system-stimulating amount of ozone gas and ultraviolet radiation, followed by administering the treated blood to a human.

A subject preferably undergoes a course of treatments, each individual treatment comprising removal of a blood aliquot, treatment thereof as described above and re-administration of the treated aliquot to the subject. A course of such treatments may comprise daily administration of treated blood aliquots for a number of consecutive days, or may comprise a first course of daily treatments for a designated period of time, followed by an interval and then one or more additional courses of daily treatments.

In one preferred embodiment, the subject is given an initial course of treatments comprising the administration of 1 to 6, more preferably 4 to 6 aliquots of treated blood. In another preferred embodiment, the subject is given an initial course of therapy comprising administration of from 2 to 4 aliquots of treated blood, with the administration of any pair of consecutive aliquots being either on consecutive days, or being separated by a rest period of from 1 to 21 days on which no aliquots are administered to the patient, the rest period separating one selected pair of consecutive aliquots being from about 3 to 15 days. In a more specific, preferred embodiment, the dosage regimen of the initial course of treatments comprises a total of three aliquots, with the first and second aliquots being administered on consecutive days and a rest period of 11 days being provided between the administration of the second and third aliquots. For optimum effectiveness of the treatment, it is preferred that no more than one aliquot of modified blood be administered to the subject per day, in one or more injection sites, and that the maximum rest period between any two consecutive aliquots during the course of treatment be no greater than about 21 days.

It may be preferred to subsequently administer additional courses of treatments following the initial course of treatments. Preferably, subsequent courses of treatments are administered following a rest period of several weeks or months, preferably at least about three weeks, after the end of the initial course of treatments. In one particularly preferred embodiment, the subject receives a second course of treatments comprising the administration of one aliquot of treated blood every 30 days following the end of the initial course of treatments, for a period of 6 months. It may also be preferred in some circumstances to follow one or more of the above-described courses of treatment by periodic “booster” treatments, if necessary, to maintain the desired effects of the present invention. For example, it may be preferred to administer booster treatments at intervals of 3 to 4 months following the initial course of treatment.

It will be appreciated that the spacing between successive courses of treatments should be such that the positive effects of the treatment of the invention are maintained, and may be determined on the basis of the observed response of individual subjects.

The present invention is a process for the treatment of or prophylaxis against acute inflammatory mammalian disorders where inappropriate cytokine expression is involved. Those disorders are generally characterized by acute inflammation that is mediated by cytokines IL-1β, IFN-γ and/or cytokines secreted from inflammatory cells e.g. Th-1 cells. A patient having such a disorder may be selected for treatment. “Treatment” includes, for example, a reduction in the number of symptoms, a decrease in the severity of at least one symptom of the particular disease or a delay in the further progression of at least one symptom of the particular disease.

One example of an acute inflammatory disorder that the process of the present invention may treat or help guard against, is acute allergic or toxic reaction from surface contact with environmental and occupational allergens or drugs through anaphylactic shock. More specific examples of such disorders include allergic contact dermatitis, acute hypersensitivity and respiratory allergy.

A second example of an acute inflammatory disorder that the process of the present invention may treat or help guard against, is acute neurological inflammatory injury such as that caused by acute infection.

A third example of an acute inflammatory disorder that the process of the present invention may treat or help guard against, is acute myocardial infarction.

Another example is prophylaxis against or treatment of acute neuronal injury resulting from cardiopulmonary bypass surgery.

A further example is prophylaxis or treatment of acute inflammatory conditions arising from surgical or medical procedures, and medically induced (“jatrogenic”) acute inflammatory conditions.

The invention may also be useful in pre-conditioning individuals about to enter an environment in which they will encounter conditions likely to lead to acute inflammatory disorder development, such as harmful chemical-containing environments and insect infested areas.

The prophylaxis or treatment methods described herein may be administered in combination with one or more other modalities. Examples of other preferred modalities include, but are not limited to, non-steroidal and steroidal anti-inflammatories. Administration in combination includes, for example, administration of the treated blood described herein, prior to, during or after administration of the other one or more modalities. One of skill in the art will be able to determine the administration schedule and dosage.

### EXAMPLE 1

Irritable contact dermatitis (ICD), or acute dermatitis, is an example of acute inflammation, in a model of which an irritant (2,4-dinitrofluorobenzene (DNFB)) is painted on the shaved skin of a mouse and then after certain time points, the draining lymph nodes are collected and analyzed for the mRNA expression of pro- and anti-inflammatory cytokines. This constitutes an accepted animal model of acute inflammatory disorder.

Balb/C mice between 6 to 8 weeks of age were assigned to two time groups, 12 hours and 24 hours. The mice were further assigned to one of 4 groups in the 24 hour group, A-D, with 5 animals in each group. Group A received no blood and no DNFB. Group B received a 50 microlitre injection of PBS and DNFB irritant treatment, but no treated blood. Group C was treated with DNFB and received an injection of 50 microlitres of untreated whole blood. Group D was treated with DNFB and received an injection of 50 microlitres of treated whole blood. The mice in the 12 hour group were assigned to one of three groups (of 5 mice per group) A-C. Group A received a 50 microlitre injection of PBS and DNFB irritant treatment, but no treated blood. Group B was treated with DNFB and received an injection of 50 microlitres of untreated whole blood. Group C was treated with DNFB and received an injection of 50 microlitres of treated whole blood. Since the negative control group A in the 24 hour group would be expected to have the same results relating to cytokine levels as the 12 hour group, only a 24 hour control group was used.

Whole blood was obtained from Balb/C mice, by extraction from a main artery through an injection needle, and treated with an anti-coagulant. An aliquot of this was subjected to the process of a preferred embodiment of the invention. The remainder was left untreated, for use in control experiments. Since these mice are genetically identical, there is not expected to be an immune response against the injected blood by the recipient mice.

To obtain treated blood, the selected aliquot, in a sterile, UV-transmissive container, was treated simultaneously with a gaseous oxygen/ozone mixture and ultraviolet light at elevated temperature using an apparatus as generally described in aforementioned U.S. Pat. No. 4,968,483 Mueller et al. Specifically, 10 ml of citrated blood was transferred to a sterile low density polyethylene vessel (more specifically, a Vasogen VC7002 Blood Container) for ex vivo treatment with stressors according to the invention. Using an apparatus described in the aforementioned Mueller patent (more specifically, a Vasogen VC7001 apparatus), the blood was heated to 42.5±1° C. and at that temperature irradiated with UV light principally at a wavelength of 253.7 nm, while oxygen/ozone gas was bubbled through the blood to provide the oxidative environment and to facilitate exposure of the blood to UV. The constitution of the gas mixture was 14.5±1.0 μg/ml, with the remainder of the mixture comprising medical grade oxygen. The gas mixture was bubbled through the aliquot at a rate of 240+24 ml/min for a period of 3 minutes.

Immediately prior to the injections, animals were anaesthetized with 0.2 ml of 5 mg/ml sodium pentobarbital via IP injection. The abdominal skin of the mouse was sprayed with 70% EtOH and a scalpel blade was used to remove about a one-inch diameter patch of hair from the abdomen. Where the mice were treated with DNFB, the shaved area was then painted with 25 μl of 0.5% DNFB in 4:1 acetone:olive oil using a pipette tip. All mice were anesthetized and had the belly area shaved. The PBS or blood (treated or untreated) was administered by injection into the lateral gastrocnemius muscle (right leg).

All animals in the two time groups were sacrificed after the respective time points. From each sacrificed animal, the draining lymph nodes were harvested. The RNA was extracted from the lymph nodes, and subjected to RT-PCR analysis for expression of the pro-inflammatory cytokines IL1-β, IFN-γ and IL-12. The results were determined in comparison with the standard reporter gene GAPDH, which is known to be expressed at 100% levels.

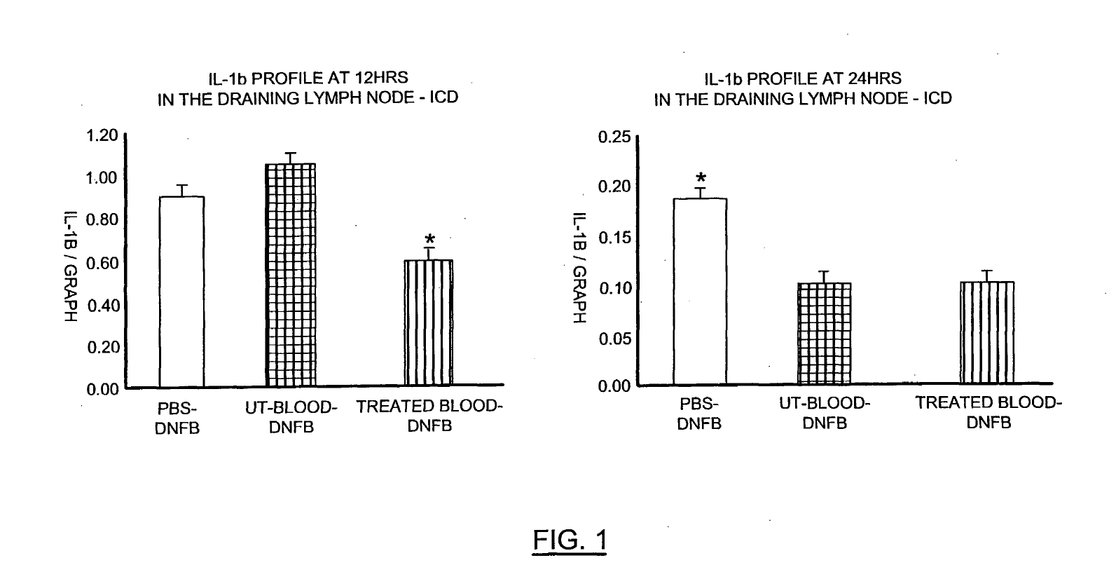

The data, as cytokine/GAPDH for the various cytokines at 12 and 24 hours, are presented graphically on FIGS. 1-3.

FIG. 1 pertains to IL-1β measurements. These are plotted, as a ratio to housekeeping gene GAPDH, as vertical axis, comparing the results for various experimental conditions at 12 hours and 24 hours. Each point represents the mean of five measurements with the error bars representing the standard error of the mean. The data at 12 hours shows the pro-inflammatory cytokine IL-1β is significantly downregulated, in comparison to the PBS-DNFB (p=0.022) and untreated blood-DNFB (p=0.001), when treated blood was injected into the mice, as determined by ANOVA. The results at 24 hours were also significant when comparing mice having the treated blood treatment in comparison to PBS-DNFB (p<0.001) and untreated blood-DNFB (p<0.001) This is an indication of the potential of the process of the present invention to combat acute IL1-β related disorders in mammalian patients, such as early pulmonary inflammation resulting from hepatic injury, unstable angina, acute juvenile and rheumatoid arthritis, and acute ischemia.

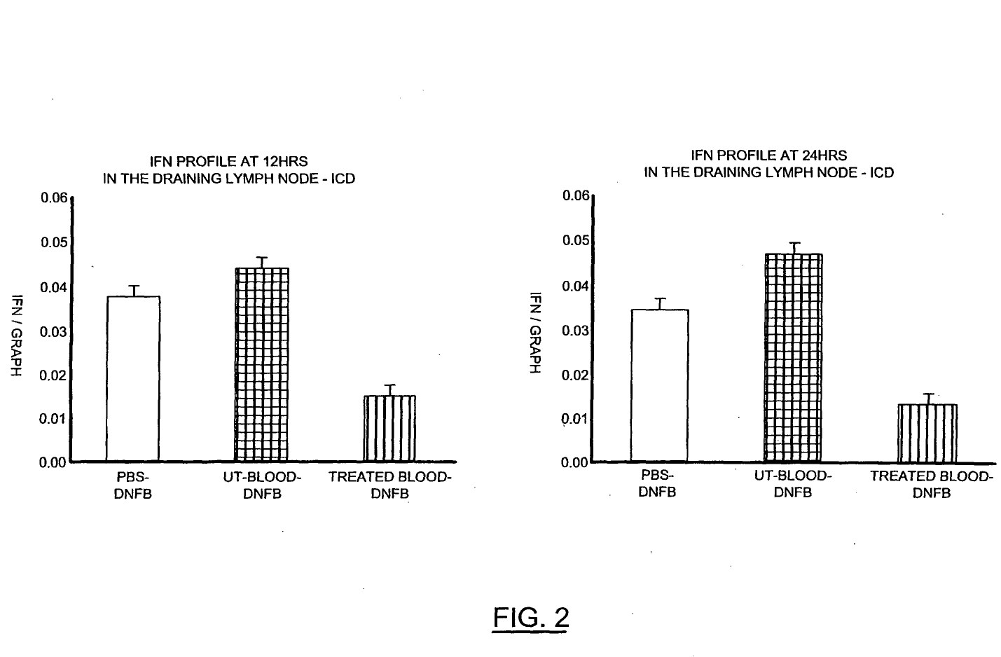

FIG. 2 similarly presents the results of measurements of IFN-γ, another pro-inflammatory cytokine. Here the effect of the treated blood is noticeable and significant at both 12 (p<0.05) and 24 hours (p<0.001) in comparison to untreated blood-DNFB or PBS-DNFB at 24 hours (p=0.011) as determined by ANOVA, further indication of the potential of this invention in treating acute inflammatory disorders, especially those in which IFN-γ plays a significant role, such as coronary arterial inflammation, pericarditis and acute coronary syndrome.

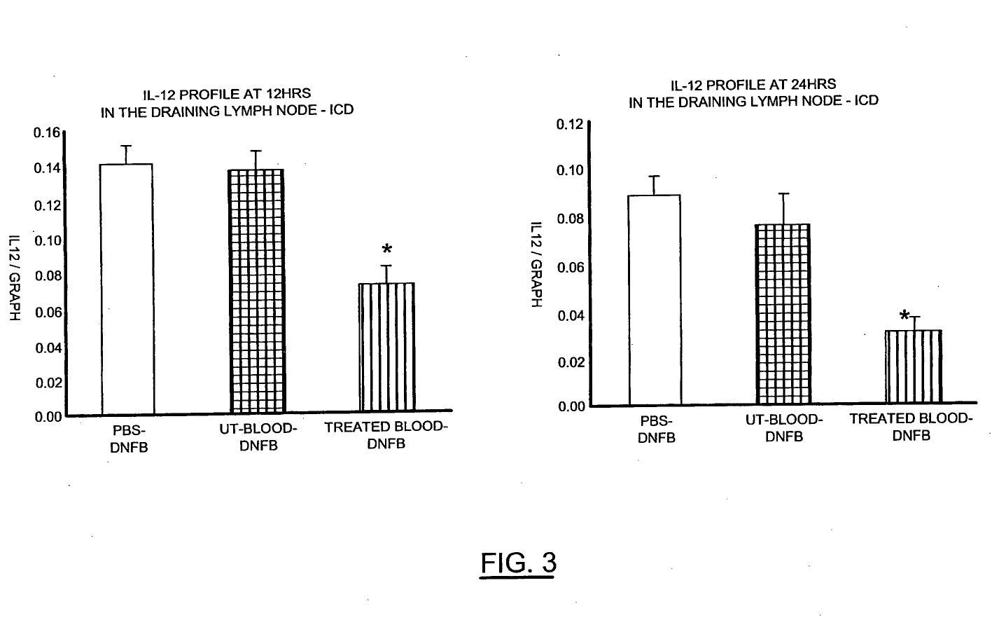

FIG. 3 similarly presents the results for measurement of IL-12, an inflammatory cytokine. Again, there is significant downregulation of IL-12 in the treated blood group at 12 hours (p=0.003) and 24 hours (p=0.003), in comparison to the PBS-DNFB condition, and significant downregulation of IL-12 at 12 hours (p=0.01) and 24 hours (p=0.01) in the untreated blood-DNFB condition as determined by ANOVA. This is indicative of the potential of the preferred embodiments of the invention in combating IL-12 related acute inflammatory disorders such as acute respiratory syndrome, acute inflammatory response due to organ transplant and acute inflammatory bowel disease.

### EXAMPLE 2

Blood (10 ml) from syngeneic male F1 Lewis Brown Norway (LBN) rats was pooled, and subjected to exposure to heat, UVC light and ozone/oxygen gaseous mixture using a VC7001 device (Vasogen Inc.). The conditions of blood treatment were as described in Example 1, namely 10 ml of blood treated with sodium citrate anticoagulant was heated to a temperature of 42.5±1° C., and at that temperature the blood was irradiated with UV light principally at a wavelength of 253.7 nm, while ozone/oxygen gas mixture (14.5±1.0 μg/ml ozone, balance medical grade oxygen) was bubbled through the blood aliquot at a rate of 240±24 ml/minute for a period of 3 minutes.

Twenty LBN male F1 rats were allotted to either a treatment group (12) or a control group (8). On day 1, day 2 and day 14, rats in the treatment group were injected in the gluteus muscle with 150 μl of the treated blood. Rats in the control group were similarly injected, on the same schedule, with 150 μl of saline.

On day 15, the animals were surgically operated on, subjecting them to coronary artery ligation. The animals were then studied by echo cardiography through the acute phase following the surgery, to monitor the cardiac function of the animals following the ligation.

The echo cardiography revealed a significant reduction in left ventricular end-diastolic area in rats of the treatment group during the acute phase, as compared with rats of the control group. This is indicative of a protective effect and early benefit on cardiac remodeling after coronary artery ligation. These results are supportive of the potential utility of the process and procedures of the present invention in treatment of acute inflammatory conditions such as acute myocardial infarction and acute inflammatory conditions arising from surgical procedures.

1-17. (canceled)

18. A method for prophylaxis or treatment of an acute inflammatory disorder, wherein the acute inflammatory disorder is characterized by a rapid onset and severe symptoms where the duration of the onset from a normal condition to one in which symptoms of inflammation are seriously manifested is up to about 72 hours, and wherein the acute inflammatory disorder involves inappropriate cytokine expression by inflammatory cells, said method comprising administering an aliquot of blood to a mammalian patient in need thereof, wherein the aliquot has been treated ex vivo with at least two stressors selected from the group consisting of an oxidizing agent, an electromagnetic emission and an elevated temperature.

19. The method of claim 18, wherein the oxidizing agent comprises ozone gas.

20. The method of claim 19, wherein the oxidizing agent comprises a mixture of medical grade oxygen and ozone gas.

21. The method of claim 20, wherein the ozone is administered as a gas stream in admixture with medical grade oxygen at a rate of from 0.01-2.0 liters per minute.

22. The method of claim 18, wherein the electromagnetic emission is ultraviolet radiation.

23. The method of claim 22, wherein the ultraviolet radiation is supplied from at least one ultraviolet lamp emitting in the C-band wavelength.

24. The method of claim 18, wherein the elevated temperature is 42.5±1° Celsius.

25. The method of claim 18, wherein three stressors are applied to the blood simultaneously, wherein the three stressors are ozone gas, ultraviolet radiation and a temperature of from 40° to 50° Celsius.

26. The method of claim 25, wherein the ozone is administered as a gas stream in admixture with medical grade oxygen at a rate of from 0.01-2.0 liters per minute.

27. The method of claim 26, wherein the blood aliquot is treated with ozone and ultraviolet radiation at a temperature from 40° to 44° Celsius for a period of from 2 to 5 minutes, the ozone/oxygen mixture being supplied at a rate of from 0.1-1.0 liters per minute, with an ozone content of from 10-300 μg/ml.

28. The method of claim 25, wherein the temperature is 42.5±1° Celsius.

29. The method of claim 18, wherein the aliquot administered to the mammalian patient is from 0.01 to 400 ml.

30. The method of claim 29, wherein the aliquot administered to the mammalian patient is from 1 to 50 ml.

31. The method of claim 18, wherein the acute inflammatory disorder is acute allergic or toxic reaction from surface contact with environmental allergen or drugs through anaphylactic shock.

32. The method of claim 18, wherein the acute inflammatory disorder is allergic contact dermatitis or acute hypersensitivity.

33. The method of claim 18, wherein the acute inflammatory disorder is acute neurological inflammatory injury.

34. The method of claim 18, wherein the acute inflammatory disorder is acute neuronal injury resulting from cardiopulmonary bypass surgery.

35. The method of claim 18, wherein the acute inflammatory disorder arises from surgical or medical procedures.

36. The method of claim 18, wherein the acute inflammatory disorder arises from medically induced actor inflammatory conditions.
